(19) United States
(12) Patent Application Publication
(10) Pub. No.: US 2016/0218552 A1
(43) Pub. Date: Jul. 28, 2016

(54) Batteries

(71) Applicant: Prash JAYARAJ, Burbank, CA (US)

(72) Inventor: Prash Jayaraj, Burbank, CA (US)

(21) Appl. No.: 14/916,150

(22) PCT Filed: Sep. 5, 2014
(86) PCT No.: PCT/US2014/054381

§ 371 Date: Mar. 2, 2016

Related U.S. Application Data
(60) Provisional application No. 61/874,301, filed on Sep. 5, 2013.
(60) Provisional application No. 61/993,987, filed on May 15, 2014.

(51) Int. Cl. H02J 7/35; H01M 2/10; A61B 90/30

(52) U.S. Cl. CPC H02J 7/355; A61B 90/30; H01M 2/1016; H01M 2220/30

(57) ABSTRACT

Described herein are batteries for medical devices as well as accessories to house and use the batteries.

### CROSS REFERENCE TO RELATED APPLICATIONS

This application claims the benefit of U.S. provisional patent application No. 61/993,987, filed May 15, 2014 and U.S. provisional patent application No. 61/874,301, filed Sep. 5, 2013, the entire disclosures of which are incorporated herein by reference.

### FIELD

Described herein are batteries and other accessories for medical devices.

### SUMMARY

Described herein are batteries used for surgical and medical tool applications. The batteries can be of any useful voltage or amperage. In some embodiments, the battery can be removable. In some embodiments, the battery can be removed from the device prior to cleaning and/or sterilization.

Some embodiments herein describe batteries for medical device lights comprising: a battery cell encased in a body section and connected to a delivery prong, wherein the body section is configured to fit within a cut away portion of a tool attachment portion and the delivery prong is configured to be inserted into a power delivery orifice.

In other embodiments, lighted surgical pencils are described comprising: surgical pencils including at least one battery powered light, a cable, and tool attachment portion; a battery cell encased in a body section and connected to a delivery prong, wherein the body section is configured to fit within a cut away portion of the tool attachment portion and the delivery prong is configured to be inserted into a power delivery orifice.

Methods are also disclosed both using the lights and lighted medical devices described herein. In one embodiment, a method of lighting a target treatment site is described comprising: attaching a battery cell encased in a body section and connected to a delivery prong to a power orifice on a tool attachment portion of a medical device at a plug end of a corded connector; and powering at least one light associated with the medical device, wherein the body section is configured to fit within a cut away portion of the tool attachment portion.

In one embodiment, power can be delivered to the medical device light from the delivery prong through a cable extending from the tool attachment portion to the medical device. The medical device can be a surgical pencil, a dental drill, an ultrasonic orthopedic needle, or the like. In some embodiments the tool attachment portion has a proximal end that plugs into an instrument and wherein the proximal end includes the cut away portion. Further, wherein when in use, the battery can be sandwiched between the tool attachment portion and the instrument.

The battery prong can be tapered to hold the battery in the tool attachment portion. The battery or each battery if more than one is used can have a capacity or capacities between about 1 mAh and about 100 mAh.

Further, in some embodiments, batteries can be configured to be removed from the tool attachment portion when the medical device is sterilized. Also, or in addition, batteries can be configured to be removed from the tool attachment portion when the medical device is discarded.

Also described herein are battery adapter systems. These systems can include: a housing configured to hold at least one battery; a cord configured to receive power from a power source; and a tube to provide suction to a medical device, wherein the tube includes at least one power cord within the tube. In some embodiments, the battery adapter systems can be configured to provide a centralized and/or easily accessible location for battery placement.

In some embodiments, the battery adapter systems can further include a second power cord to supply power from the battery to the medical device. Further, the housing can include a vacuum source connection portion configured to attach to a vacuum source.

In some embodiments, the housing includes a door to cover the at least one battery. The housing can be formed of a thermal set rigid plastic. Further still, the housing can have a bimodal shape including an elongated first end and a circular second end, wherein the elongated first end is configured to receive or be produced with tubing attached and wherein the circular second end is configured to provide the power cable.

Other embodiments herein describe an adapter system including: a housing including a proximal end including three prongs; a distal end including a first input, a second input, a third input and a forth input; and at least one battery. In some embodiments, the adapter system can be configured to reside between a supply source and a modified tool interface portion.

The adapter system can be configured to convert a conventional supply source socket which includes three inputs into the first input, the second input, the third input, and the forth input. The third input can be configured to supply power to an accessory on a medical device attached the adapter system.

The adapter system can further include a printed circuit board configured to convert power from a supply source to a lower power needed by an accessory. Also, the adapter system can include one or more solar cells configured to charge the at least one battery.

### BRIEF DESCRIPTION OF THE DRAWINGS

FIG. 1 illustrates a perspective view of a medical tool attachment portion including a battery.

FIG. 2 illustrates a back view of a medical tool attachment portion including a battery.

FIG. 3 illustrates a front view of a medical tool attachment portion including a battery.

FIG. 4 illustrates a side view of a medical tool attachment portion including a battery.

FIG. 5 illustrates a top view of a medical tool attachment portion including a battery.

FIG. 6 illustrates a bottom view of a medical tool attachment portion including a battery.

FIG. 7 illustrates an exploded view of a medical tool attachment portion including a battery.

FIG. 8 illustrates an exploded view of another medical tool attachment portion including a different battery.

FIG. 9 illustrates an exploded view of another medical tool attachment portion including still a different battery.

FIG. 10 illustrates an exploded view of another medical tool attachment portion including a battery attachable to the back of the medical tool attachment portion.

FIG. 11 illustrates a surgery utilizing a medical tool with a battery attached to the attachment portion.

FIG. 12 illustrates a medical tool attachment portion of FIG. 1 attached to a supply instrument.

FIG. 13 illustrates an exploded view of medical tool attachment portion of FIG. 1 attachable to a supply instrument.

FIG. 14A illustrates a battery adapter system as described herein. FIG. 14B illustrates a cross-section of the tubing.

FIG. 15 illustrates an adapter system as described herein.

### DETAILED DESCRIPTION

As technology continues to evolve, more and more devices, particularly medical; devices rely on electrical power to function. With almost any electronic device, the freedom from a cord(s) or even an additional cord such as a power cord may be desired.

In one embodiment, described herein, batteries can be used to power a light on, in or otherwise associated with a medical device. In some embodiments, the medical device can be a surgical tool. With any medical procedure or surgical procedure, an ability to properly visualize the target site can be vital. Often times, illumination of a target site can be difficult to achieve and the batteries and systems described herein can aid in achieving illumination of a target site.

A well lit surgical site can provide significant advantages to patient and physician comfort as well as to a surgeon's or medical practitioner's stamina. This is because a lighted target site, if desired, can allow surgery to take place under ambient light conditions without sacrificing complete and accurate visualization of the target surgical field. These capacities can be beneficial for a surgeon and surgical team working long hours under bright lights that generate intense heat in order to visualize the target surgical area. These bright lights generating intense heat can result in previously unavoidable surgeon discomfort and fatigue. Additionally, it is not uncommon for a surgeon to be wearing several layers of clothing along with surgical barriers, including gloves, face barriers, goggles, hats, and overcoats, to name a few, during a given surgical procedure, further contributing to the discomfort and fatigue normally associated with hot and bright surgical working environments.

Compounding matters, the complexity of contemporary operating rooms has increased over the years as a result of the extra equipment, additional electronic devices, fixtures, associated power cords and the like required for ever more complicated surgeries. Such situations are not conducive to comfortable, non-fatiguing surgical environments. The ease of use and the optional ability to use ambient lighting when using the described battery powered devices can address these issues.

As an additional benefit, the ambient lighting conditions that now can be utilized without sacrificing visualization may also reduce reflected glare and high contrast shadows in the surgical environment that, in the past, could confuse or possibly even overwhelm the surgeon's vision. Using a surgical tool with a light powered by the herein described batteries, a surgeon, surgical team, and or audience (e.g., students) may no longer be required to position themselves out of certain areas in order to reduce shadows that they might cast on the target surgical site. A battery powered, lighted surgical device can addresses this problem by reducing surgical theater lighting thereby reducing shadows and increasing visibility, especially of the target site.

Similarly, it is not uncommon for a surgeon to look away from a target surgical site in order to change or to move equipment, to take a mental break, or to communicate with a surgical team or students. Upon looking back onto the traditional target surgical site, the surgeon would have to wait briefly to allow his eyes to adjust to the normal high intensity lighting, causing delays in the procedure. The present battery powered, lighted surgical devices can eliminate this problem under ambient light conditions while still providing effective visualization of the surgical site.

The use of ambient light in medical or surgical processes and the resulting reduced heat and complexity in the operating room also adds to the comfort of a surgical patient and enhances the compliance of the patient with the needs of the surgeon. Patient comfort during a surgical procedure can be very important, especially when the patient is under local anesthesia and is conscious. It is not uncommon for bright lights to be focused on at least a portion of a patient, typically on the target surgical site. Such lighting systems can get hot and make a patient uncomfortable. Patients who are uncomfortable can be more on edge, squirm and/or twitch, or can be tense. The battery powered, lighted surgical devices' ambient light capabilities can simplify and shorten a medical procedure, provide enhanced patient comfort and compliance, and improve the medical procedure's outcome.

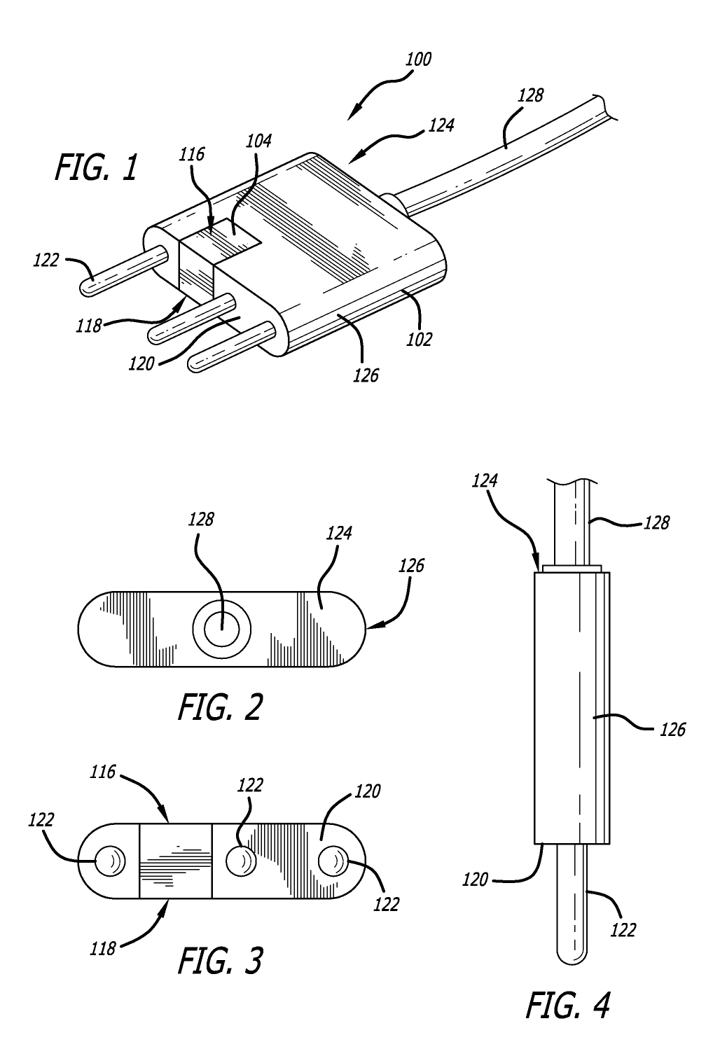

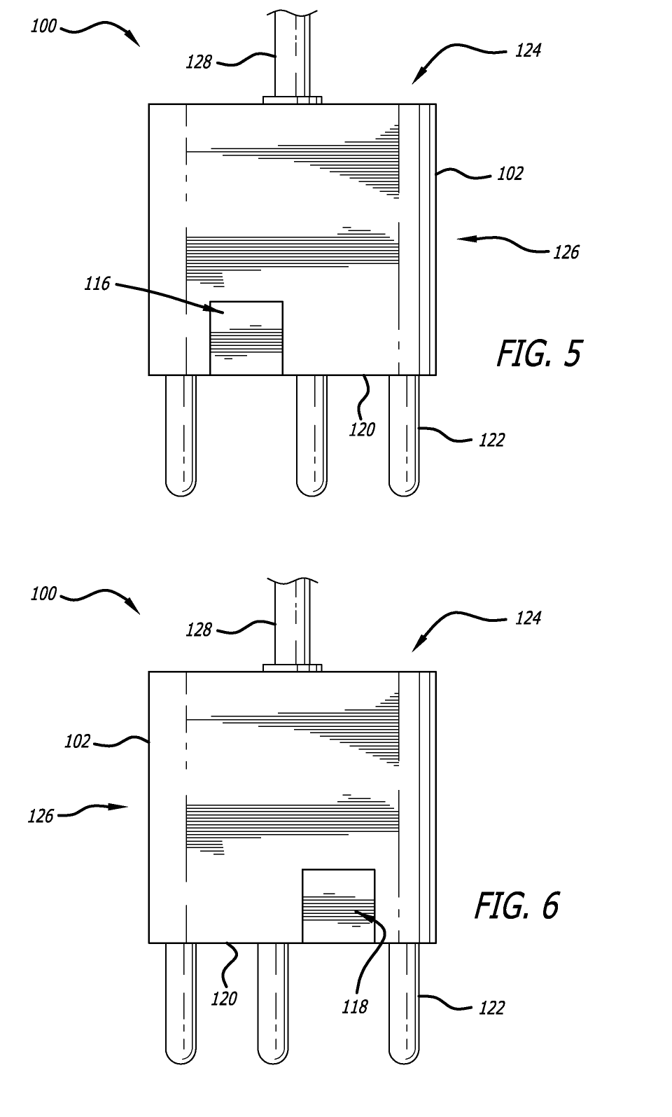

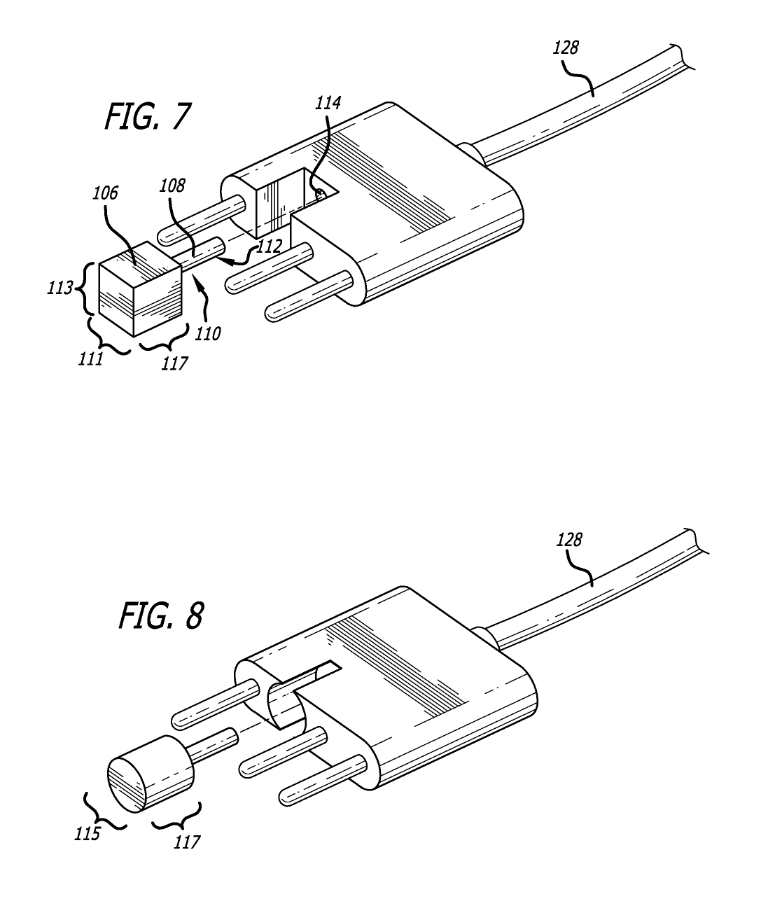

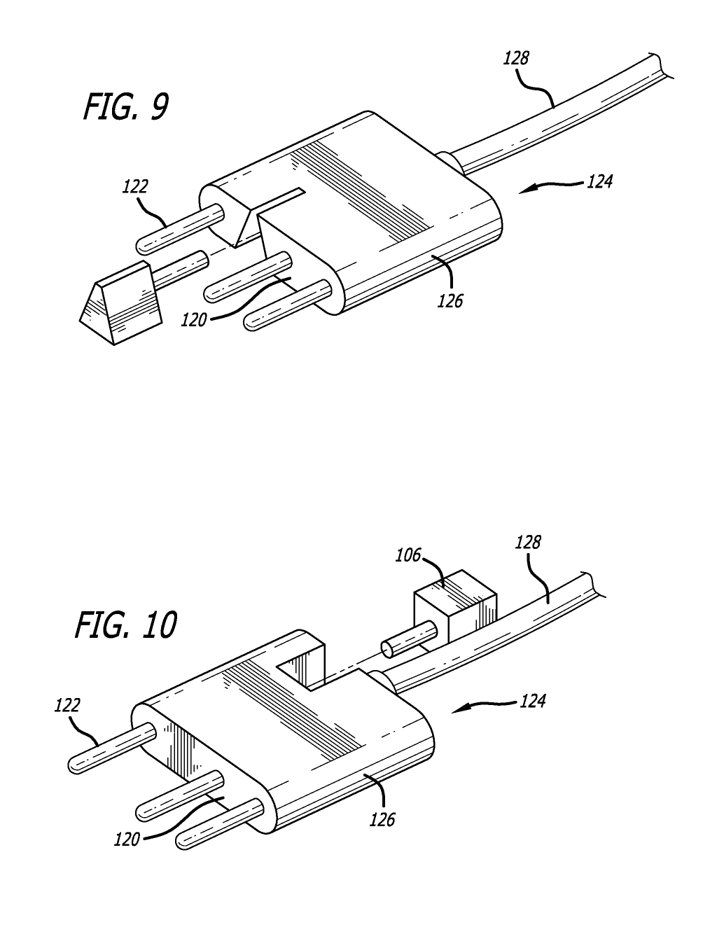

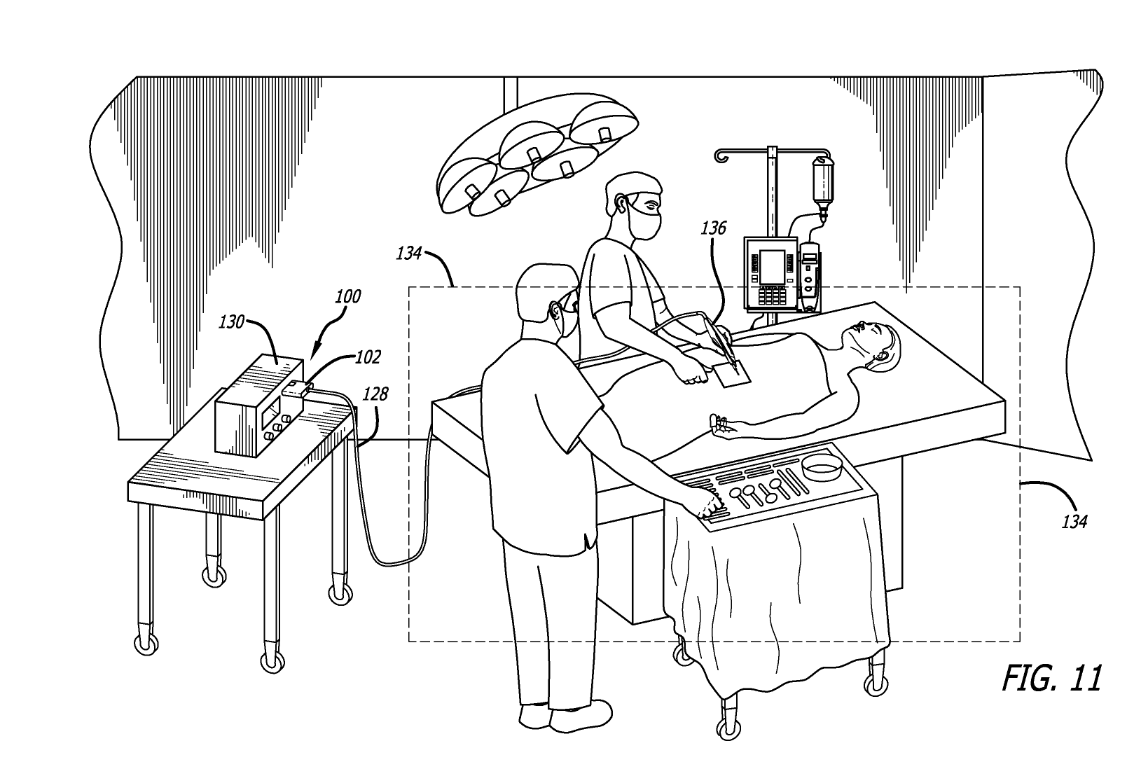

In one embodiment, at least one battery can be housed within, on, or otherwise associated with a surgical tool, such as on an attachment portion. For example, battery system 100 illustrated in FIG. 1 includes tool interface portion 102 and battery 104.

In some embodiments, power can be provided by a battery that is charged by a solar panel, solar cell, or other electrochemical cell. A solar cell can be placed on the medical device itself or on the tool attachment portion 102. Preferably, a solar cell or solar cells can be positioned on the device or tool attachment portion 102 where light is likely to hit and charge a battery. In some embodiments, a solar cell need not power a battery, but may directly power a feature such as a light or display.

Tool attachment portion 102 can be, but is not limited to, a power plug for powering other aspects of the device, a data connection, a mechanical connection, or a combination thereof. In one embodiment, tool attachment portion 102 can be a power plug for a surgical pencil. In another embodiment, tool attachment portion 102 can be a mechanical attachment for a surgical drill or saw.

The batteries can be of any useful voltage or amperage. The battery can be of any design, such as, but not limited to galvanic cells, electrolytic cells, fuel cells, flow cells, voltaic piles, photonic cells, and the like. The battery can be of any chemistry, such as, but not limited to alkaline, carbon-zinc, Li—FeS₂, NiMH, NiCd, Li, Li polymer, zinc-air, mercury, copper, manganese, mercuric-oxide, silver, silver oxide, Li-ion, or combinations thereof. Other common battery types not listed are considered within the scope of batteries than can be used herein. The battery can be a wet cell or dry cell battery. Batteries can be single use or rechargeable.

Batteries can have capacities of about 1 mAh, about 2 mAh, about 3 mAh, about 4 mAh, about 5 mAh, about 10 mAh, about 15 mAh, about 20 mAh, about 25 mAh, about 30 mAh, about 35 mAh about 40 mAh, about 45 mAh, about 50 mAh, about 55 mAh, about 60 mAh, about 65 mAh, about 70 mAh, about 75 mAh, about 80 mAh, about 85 mAh, about 90 mAh, about 100 mAh, about 200 mAh, about 300 mAh, about 400 mAh, about 500 mAh, about 600 mAh, about 700 mAh, about 800 mAh, about 900 mAh, about 1,000 mAh, at least about 1 mAh, at least about 5 mAh, at least about 10 mAh, at least about 15 mAh, at least about 20 mAh, between about 1 mAh and about 100 mAh, between about 1 mAh and about 500 mAh, between about 1 mAh and about 1,000 mAh, between about 10 mAh and about 100 mAh, between about 5 mAh and about 50 mAh, between about 50 mAh and about 300 mAh, or any value or range encompassed by or between these values.

In some embodiments, a battery can have a shape that compliments a portion of tool attachment portion 102. For example, as illustrated in FIGS. 1-5, battery 104 can include a body section 106 and at least one peg 108. In some embodiments, battery 104 can include two, three, four, five, six, seven, eight or more pegs 108.

In other embodiments, peg 108 can have a generally cylindrical shape or have a circular cross-section with a taper 110 at its distal end 112. This taper can allow battery to fit snugly within tool attachment portion 102. Although a cylindrical peg is illustrated, any cross section can be used. Which ever cross section is used, plug orifice 114 can have a shape that compliments it and in some embodiments, secures peg 108.

In some embodiments, battery 104 need not include a peg 108. Rather, battery 104 can include two or more contact points and one or more features on body section 106 that hold or secure battery 104 to tool attachment portion 102. In other embodiments, body section 106 can include two or more contact points and battery 104 can include one or more features that hold or secure it to tool attachment portion 102. In these arrangements, battery 104 can be connected to tool attachment portion 102 or the tool itself using a friction fit, a snap, a locking snap feature, a screw type fitting including threads, and the like.

Body section 106 can have a square or rectangular cross-sectional body portion that compliments a square or rectangular opening in tool attachment portion 102. In other embodiments, body section 106 can have a circular, triangular, trapezoidal, torx, star, hexagonal, octagonal, pentagonal, substantially circular, or rectilinear cross-section.

If square or rectangular, width 111 of body section 106 can be about 1 mm, about 2 mm, about 3 mm, about 4 mm, about 5 mm, about 6 mm, about 7 mm, about 8 mm, about 9 mm, about 10 mm, at most about 12 mm, at most about 10 mm, between about 1 mm and about 10 mm, or between about 5 mm and about 10 mm. Also, if square or rectangular, height 113 of body section 106 can be matched to the height of tool attachment portion 102 or about 1 mm, about 2 mm, about 3 mm, about 4 mm, about 5 mm, about 6 mm, about 7 mm, about 8 mm, about 9 mm, about 10 mm, at most about 12 mm, at most about 10 mm, between about 1 mm and about 9 mm, or between about 5 mm and about 9 mm.

If circular or other substantially circular or non-rectilinear shape, the largest dimension or diameter 115 of body section 106 can be about 1 mm, about 2 mm, about 3 mm, about 4 mm, about 5 mm, about 6 mm, about 7 mm, about 8 mm, about 9 mm, about 10 mm, at most about 12 mm, at most about 10 mm, between about 1 mm and about 9 mm, or between about 5 mm and about 9 mm.

Body section 106 can have a depth 117 of about 1 mm, about 2 mm, about 3 mm, about 4 mm, about 5 mm, about 6 mm, about 7 mm, about 8 mm, about 9 mm, about 10 mm, at most about 12 mm, at most about 10 mm, between about 1 mm and about 10 mm, or between about 5 mm and about 10 mm.

When installed into orifice 114, body section 106 can have at least one portion accessible for gripping to remove battery 104 from tool attachment portion 102. For example, as illustrated in FIG. 1, first access surface 116 and second access surface 118 can be used to grip and remove battery 104 from tool attachment portion 102. The at least one portion accessible for gripping can have a surface that aids in gripping. For example, the surfaces can be rough or another type of non-skid surface such as a perforated surface.

In another embodiment, tool attachment portion 102 and body section 106 can include complimentary keying surfaces such as, for example, a ridge(s) that prevent inserting an incorrect battery or from inserting a battery at an improper orientation. In some embodiments, the keying surface and the cross-sectional shape of body section 106 can be the same. In other embodiments, body section 106 can have a cross-sectional shape and independently have one or more keying surfaces.

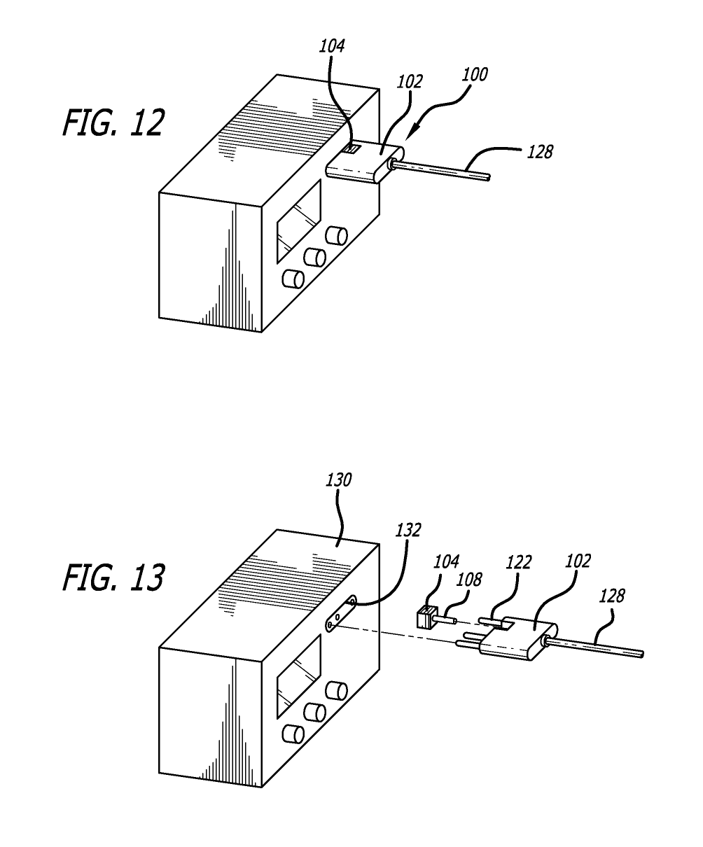

In one embodiment, battery 104 can be housed on proximal face 120 of tool attachment portion 102. Such a configuration can allow the battery at least partially to be held in place when tool attachment portion 102 is plugged in or attached to another device or supply source. For example, in the case of FIGS. 12 and 13, tool attachment portion 102 includes three prongs 122 that plug into a supply source 130 such as a power generator or amplifier. When tool attachment portion 102 is plugged into supply source 130, battery 104 becomes non-removable because it is sandwiched between tool attachment portion 102 and supply source socket 132. In the case of tool attachment portion 102 being screw threaded or friction fitted onto another device, battery can be configured such that it becomes sandwiched between tool attachment portion 102 and the device being attached to.

In other embodiments, battery 104 can be configured to be installed on the distal face 124 of tool attachment portion 102. In still other embodiments, battery 104 can be configured to be installed on a transverse face 126 of tool attachment portion 102. For example, battery 104 can be installed on the transverse face within view in FIG. 1 or on the transverse face not within view in FIG. 1. In still other embodiments, battery 104 can be configured to be installed on the top face of tool attachment portion 102 or on the bottom face of tool attachment portion 102.

In some embodiments, battery 104 can be plugged into an orifice on the medical tool or surgical instrument body. In an example embodiment, the battery is placed at a location that is not disrupted by use of the instrument or tool.

Power from battery to a light or other electronic component can be transmitted through a cable, for example, cable 128. Cable 128 can independently transmit power from the battery to the light or other electronic component or can include other power, data or mechanics. In one embodiment, the tool is a surgical pencil and the cable transmits power to the cutting tip and separately power from the battery to a light on the surgical pencil.

In some embodiments, sterilization of a medical device or surgical tool can be desired. Sterilization can be particularly important on reusable devices. The batteries described herein can be designed to be removable from a device when sterilization is desired. Removal of a battery from a device prior to sterilization can be important because some sterilization techniques can damage the battery, or in some cases even explode the battery. Not only can this be dangerous, it can also destroy the device being sterilized.

For example, in the device illustrated in FIG. 1, battery 104 can be simply removed from tool attachment portion 102, and the device can be sterilized.

Sterilization can include any common form of cleaning or sterilization, such as, but not limited to washing with soap, washing with alcohol, seam sterilization, electron beam sterilization, autoclave, gamma irradiation, combinations thereof and the like.

Battery 104 can be made of materials that can be washed and sanitized. In some embodiments, battery 104 can be made air tight. Even if battery 104 is not cleaned or sterilized, because battery 104 is connected to orifice 114 on tool attachment portion 102, it can be situated outside the sterile field of an operating room where the plug is plugged in.

As such, in some embodiments, a device can be cleaned, sterilized, and packaged prior to delivery to an operating room. Then, once the procedure is to begin, the device in unsealed, the cord unwound, a battery inserted into a tool attachment portion, and plugged in to a supply source 130. In this scenario, tool attachment portion 102 is outside sterile filed 134. Once plugged in, a battery powered feature on medical device 136 can be activated or turned on. In one embodiment, medical device 136 can be a surgical pencil and battery powered feature can be a light.

Outside the sterile field can be about 2 ft, about 3 ft, about 4 ft, about 5 ft, about 6 ft, about 7 ft, about 8 ft, about 9 ft, about 10 ft, about 12 ft, about 15 ft, at least about 2 ft, at least about 4 ft, at least about 6 ft, or at least about 8 ft away from a patient.

In some embodiments, the medical device or surgical tool can be separated for disposal. Whether a single use device or a multiuse device, at some point, it needs to be disposed of. In order to dispose of a device including a battery, the disposal must meet both biohazard standards and electronic waste standards. In some embodiments, a device may not need to meet biohazard standards. In any case, if the battery can be simply removed prior to disposal, the device can be discarded simply in a biohazard container or a trash can if applicable. The battery can be discarded in an appropriate electronic waste bin. Separating the electronic waste battery from a device can cut cost of disposal.

In some embodiments, a tool attachment portion can be configured to accept standard sized batteries. Standard size batteries can include, but are not limited to round, cylindrical batteries such as AA, AAA, AAAA, C, D, and button cell (such as lithium button), coin cell, and non-round batteries such as 4.5V box and 9V box batteries, and the like. Further button cell or coin cell batteries can be used. Batteries can be removed as needed to clean and/or sterilize the device prior to use.

In some embodiments, battery 104 can be rechargeable. A medical office and/or operating theatre recharging station can be provided. A charging station can charge one or more batteries 104 as needed and be conveniently placed for access to charged batteries as needed.

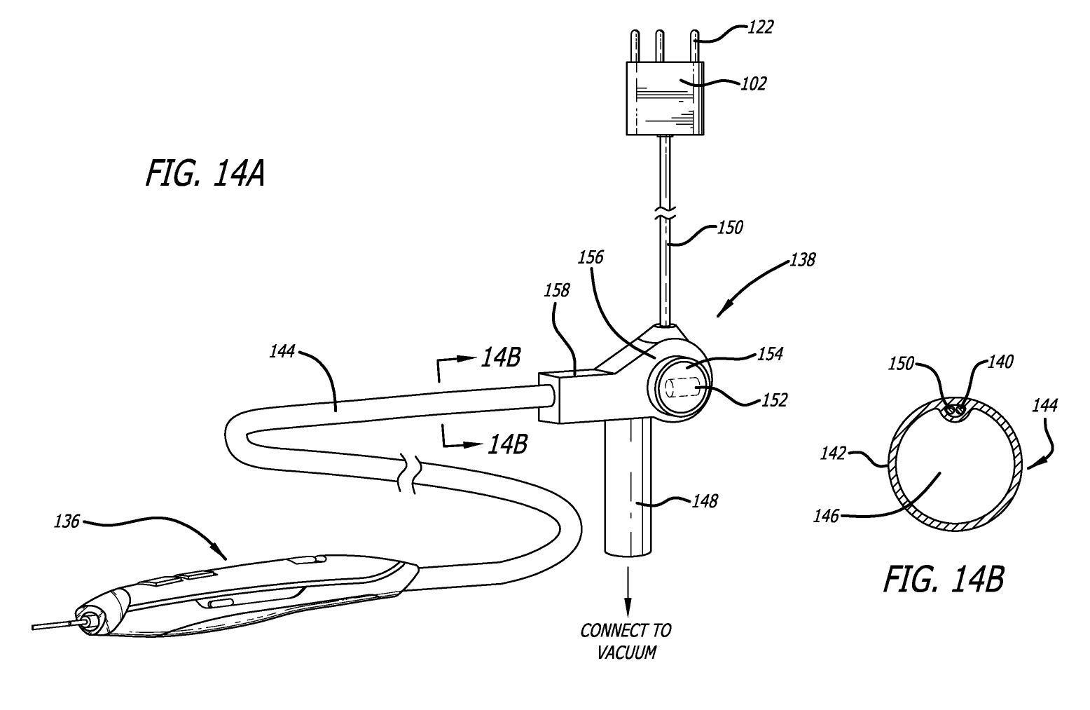

In one embodiment, a battery adapter system can be provided. As illustrated in FIGS. 14 A and 14B, battery adapter system 138 can be configured to provide a centralized and easily accessible location for battery placement which can be located outside sterile filed 134. Battery adapter system 138 can be configured to provide battery power to medical device 136 though a cable 140 housed within wall 142 of tubing 144. In other embodiments, cable 140 can be separate from tubing 144.

Tubing 144 can have an internal lumen 146 that can be used to supply suction to medical device 136. Thus, battery adapter system 138 can include a vacuum source connection portion 148. Vacuum source connection portion 148 can be configured to attach to any conventional vacuum source or medical grade vacuum source. In one embodiment, vacuum source connection portion 148 can attach to a vacuum canister such as a suction canister.

Tubing 144 can further include a second cable 150 within wall 142 of tubing 144. Second cable 150 can provide cauterizing power to medical device 136 and can ultimately terminate at tool attachment portion 102.

Battery adapter system 138 can include a removable battery or batteries. Battery 152 can be the same or different than battery 104. Battery 152 can be located behind door 154. Door 154 can be hinged to housing 156, screwed onto hosing 156, snapped onto housing 156 or the like.

In one embodiment, battery 152 can be located in housing 156 in order to avoid interference from supply source 130.

Battery adapter system 138 can be permanently attached to medical device 136. In other embodiments, battery adapter system 138 can be removable from medical device 136, for example, from a barbed fitting on medical device 136 or from a barbed or other connection on housing 156.

As a system or kit, battery adapter system 138 can include housing 156, tubing 144, adapter 148, cable 150, and tool attachment portion 102. In other embodiments, as a system or kit, battery adapter system 138 can include housing 156, adapter 148, cable 150, and tool attachment portion 102. In still other embodiments, as a system or kit, battery adapter system 138 can include housing 156, tubing 144, adapter 148, cable 150, medical device 136, and tool attachment portion 102. The kits may or may not include instructions for use.

In other embodiments, battery 152 can be permanently attached to battery adapter system 138. For example, a battery or batteries can be installed in housing 156 and then door 152 can be glued onto housing 156, welded onto housing 156, or the like.

Battery 152 can further be located along tubing 144. Tubing 144 can include a user replaceable battery at any location along its length. In other embodiments, a battery along tubing 144 may not be user replaceable, but be permanently attached thereto.

Housing 156 can be formed of any appropriate medical grade material. In other embodiments, housing 156 need not be formed of a medical grade material. In some embodiments, the material is a polymer or mixture of polymers. The polymer or combination of polymers chosen to form housing 156 must be rigid enough to hold a particular configuration and perform its intended function. In some example embodiments, the polymer used is a thermal set rigid plastic.

In some embodiments, housing 156 can have a bimodal shape including an elongated first end 158 and a circular second end 160. Elongated first end 158 can be configured to receive or be produced with tubing 144 attached thereto. Circular second end 160 can be configured to provide cable 150. In some embodiments, these two ends can be reversed.

Battery adapter system 138, in one embodiment, can be reusable. In such a case, the systems are washable and sterilizable using conventional sterilization techniques. For example, battery adapter system 138 may be sealed sufficiently to allow multiple washings with a detergent or alcohol based cleaner without damaging the system. Further, for example, the systems can be sterilized using at least one sterilization technique including, but not limited to gamma irradiation, pressure sterilization and/or steam sterilization.

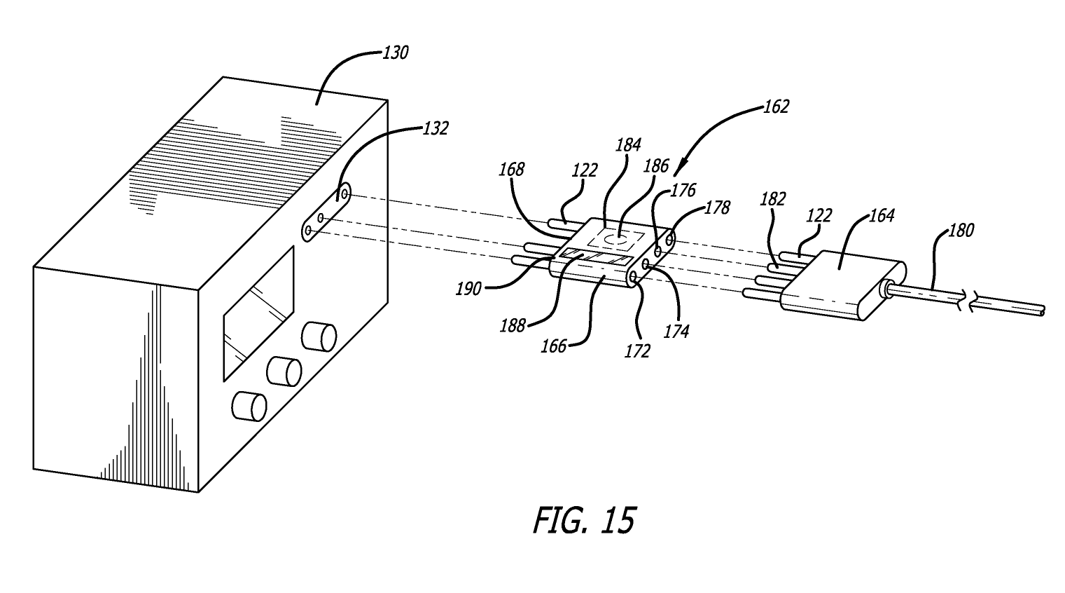

In another embodiment, an adapter system 162 is illustrated in FIG. 15. Adapter system 162 can be configured to reside between supply source 130 and a modified tool interface portion 164. Adapter system 162 can include an adapter housing 166 that can have a generally or substantially similar shape as tool interface portion 102 described herein. Proximal end 168 can be configured to supply three prongs 122 commonly found on tool interface portion 102. Distal end 170 can include a first input 172, a second input 174, a third input 176 and a forth input 178. Thus, adapter system 162 can convert a conventional supply source socket 132 which includes three inputs into first input 172, second input 174, third input 176 and forth input 178. The new input, third input 176, can be configured to supply power to an accessory on a medical device attached to cord 180. For example, in some embodiments, that accessory can be one or more lights.

Modified tool interface portion 164 includes the standard three prongs 122 plus an additional fourth prong 182 that aligns to insert into third input 176 of adapter system 162. This fourth prong 182 accepts power to provide it to the at least one accessory. Modified tool interface portion 164 can have a generally or substantially similar shape as tool interface portion 102 described herein.

Medical devices 136 can be provided with modified tool interface portion 164 as well as an adapter system 162 in order to allow a user to hook the medial device 136 to supply source socket 132 on a conventional supply source 130.

Adapter system 162 can include a printed circuit board 184 that can convert power from supply source 130 to the lower power needed by an accessory such as a light. For example, printed circuit board 184 can include one or more resistors, capacitors, and the like that can appropriately convert power as needed.

Further, adapter system 162 can include one or more battery 186. Battery 186 can be the same or different than battery 104 or battery 152. Battery 186 can act alone to power an accessory through fourth input 176 or can act in concert with printed circuit board 184 to provide power as needed.

Battery 186 can be configured to be inserted into any surface or any face of adapter housing 166 as described in other embodiments herein. For example, battery 186 can be configured to be inserted into a proximal face, a distal face, a side face, a top face, or a bottom face of adapter housing 166.

In some embodiments, battery 186 can be permanently built into adapter housing 166. Even if permanently built into adapter housing 166, adapter housing 166 can be configured to be broken open to allow proper disposal of battery 186. Once broken open, adapter housing 166 may no longer be functional and must be disposed. In other embodiments, adapter housing 166 can be re-assembled for further use with a new battery.

Further still, adapter system 162 can include one or more solar cell 188. Solar cell 188 can reside on any face of adapter housing 166. However, in some embodiments, a solar cell(s) 188 can be located on top face 190 in order to acquire light when plugged into supply source 130. The solar cells can act in concert with printed circuit board 184 to provide power as needed. In other embodiments, solar cell 188 may be configured to charge battery 186.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A battery for a medical device light comprising:
a battery cell encased in a body section and connected to a delivery prong,
wherein the body section is configured to fit within a cut away portion of a tool attachment portion and the delivery prong is configured to be inserted into a power delivery orifice.

2. The battery of claim 1, wherein power is delivered to the medical device light from the delivery prong through a cable extending from the tool attachment portion to the medical device.

3. The battery of claim 1, wherein the medical device is a surgical pencil, a dental drill, an ultrasonic orthopedic needle, or the like.

4. The battery of claim 1, wherein the body section has a square, rectangular, elliptical, triangular, rectilinear, trapezoidal, or torx cross section.

5. The battery of claim 1, wherein the tool attachment portion has a proximal end that plugs into an instrument and wherein the proximal end includes the cut away portion.

6. The battery of claim 5, wherein when in use, the battery is sandwiched between the tool attachment portion and the instrument.

7. The battery of claim 1, wherein the delivery prong is tapered to hold the battery in the tool attachment portion.

8. The battery of claim 1, wherein the battery has a capacity of between about 1 mAh and about 100 mAh.

9. The battery of claim 1, wherein the battery is configured to be removed from the tool attachment portion when the medical device is sterilized.

10. The battery of claim 1, wherein the battery is configured to be removed from the tool attachment portion when the medical device is discarded.

11. A lighted surgical pencil comprising:
a surgical pencil including at least one battery powered light, a cable, and tool attachment portion;
a battery cell encased in a body section and connected to a delivery prong,
wherein the body section is configured to fit within a cut away portion of the tool attachment portion and the delivery prong is configured to be inserted into a power delivery orifice.

12. The lighted surgical pencil of claim 11, wherein power is delivered to the at least one battery powered light from the delivery prong through the cable extending from the tool attachment portion to the surgical pencil.

13. The lighted surgical pencil of claim 11, wherein the body section has a square, rectangular, elliptical, triangular, rectilinear, trapezoidal, or torx cross section.

14. The lighted surgical pencil of claim 13, wherein the body section has a square cross section.

15. The lighted surgical pencil of claim 11, wherein the tool attachment portion has a proximal end that plugs into an instrument and wherein the proximal end includes the cut away portion.

16. The lighted surgical pencil of claim 15, wherein when in use, the battery is sandwiched between the tool attachment portion and the instrument.

17. The lighted surgical pencil of claim 11, wherein the delivery prong is tapered to hold the battery in the tool attachment portion.

18. The lighted surgical pencil of claim 11, wherein the battery has a capacity of between about 1 mAh and about 100 mAh.

19. The lighted surgical pencil of claim 11, wherein the battery is configured to be removed from the tool attachment portion when the medical device is sterilized.

20. The lighted surgical pencil of claim 11, wherein the battery is configured to be removed from the tool attachment portion when the medical device is discarded.

21-44. (canceled)
